(19) United States
(12) Patent Application Publication
(10) Pub. No.: US 2014/0220036 A1
(43) Pub. Date: Aug. 7, 2014

(54) METHODS FOR TREATING NEURODEGENERATIVE DISEASES AND FOR IDENTIFYING AGENTS USEFUL FOR TREATING NEURODEGENERATIVE DISEASES

(71) Applicants: Rodolfo R. Llinas, New York, NY (US); Herman Moreno, New York, NY (US); Mutsuyuki Sugimori, New York, NY (US)

(72) Inventors: Rodolfo R. Llinas, New York, NY (US); Herman Moreno, New York, NY (US); Mutsuyuki Sugimori, New York, NY (US)

(21) Appl. No.: 13/756,743

(22) Filed: Feb. 1, 2013

(51) Int. Cl. A61K 39/395; A61K 31/7088; G01N 33/543

(52) U.S. Cl. CPC A61K 39/3955; G01N 33/543; A61K 31/7088
USPC 424/172.1; 506/9; 514/44 R

(57) ABSTRACT

The present invention provides methods of inhibiting a tau protein such as h-tau₄₂ or a biologically active fragment, derivative or analog thereof, methods of treating a disease caused by a tau protein such as h-tau₄₂, and methods to identify agents that may inhibit a tau protein such as h-tau₄₂. The methods for identifying an agent effective to inhibit a tau protein may feature administering an agent; and observing either i) a reduction in biological activity of the tau protein or a biologically active fragment, derivative or analog thereof or ii) a reduction in phosphorylation of the tau protein or a biologically active fragment, derivative or analog thereof.

### FIELD OF THE INVENTION

The present invention relates to methods for identifying agents useful for treating neurodegenerative diseases, agents identified as useful for treating such, and methods for treating neurodegenerative diseases.

### BACKGROUND OF THE INVENTION

Classical neuropathological studies characterized the intracellular accumulation and aggregation of abnormal filaments composed primarily of the microtubule associated protein tau as a hallmark for a variety of neurodegenerative disorders known as tauopathies, as exemplified by progressive supranuclear palsy, Pick's disease, corticobasal degeneration, and frontotemporal dementia with Parkinsonism linked to chromosome 17 (FTDP-17) (Lee, et al., Annu. Rev. Neurosci. 2001; 24:1121-1159). The most common tauopathy, Alzheimer's disease (AD), is also characterized by additional filamentous structures paired helical filaments (PHFs) and straight filaments (SFs). These filaments eventually form large aggregations, known as neurofibrillary tangles (NFTs). In addition, diffuse and mature senile plaques, which are predominantly composed of amyloid beta (Aβ) peptides, are present in AD brains too (Selkoe, Physiol. Rev. 2001; 81: 741-766).

A number of studies have investigated the role of different forms of Aβ peptides and aggregates in synaptic function (Arancio, et al., EMBO J. 2004; 23: 4096-4105; Moreno, et al., Proc. Natl. Acad. Sci. U.S.A. 2009; 106: 5901-5906), but so far the role of tau or tau aggregates in neurotransmission has not been elucidated.

The association between tau filaments, neuron loss, and brain dysfunction in vertebrates and invertebrates originally led to the hypothesis that NFTs invariably cause brain dysfunction and neurodegeneration. However, mouse tauopathy studies indicate that severe abnormalities in synaptic function can precede neuronal loss and even NFTs formation (LaFerla, et al., Nat. Rev. Neurosci. 2007; 8: 499-509; Yoshiyama, et al., Neuron 2007; 53: 337-351). The molecular mechanisms responsible for this early malfunction (as well as those responsible for tau polymerization dependent pathogenesis) remain unknown (Marx, Science 2007; 316: 1416-1417). Neurofibrillary degeneration is accompanied by lysosomal hypertrophy (Nixon, et al., Ann. N.Y. Acad. Sci. 1992; 674: 65-88), beading and degeneration of distal dendrites (Braak, et al., Acta Neuropathol. 1994; 87: 554-567; Marx, Science 2007; 316: 1416-1417) and axonal damage (Kowall, et al., Ann. Neurol. 1987; 22: 639-643).

Previous experiments in drosophila have shown that misexpression of human-tau (h-tau), the same isoform as the one used in the presently described studies, produced significant neurodegeneration (Jackson, et al., Neuron 2002; 34: 509-519; Avila, et al., Physiol. Rev. 2004; 84: 361-384; Steinhilb, et al., Mol. Biol. Cell 2007; 18: 5060-5068). In the drosophila model tau co-expressed with Shaggy, which generated a single fly homolog of GSK-3β, the phenotype was aggravated (Jackson, et al., Neuron 2002; 34: 509-519). Dysfunctional phenotypes were also found in the central neurons of lamprey, where long-term expression (2-38 days) of several h-tau isoforms produced neurodegenerative changes as a result of accumulation of h-hyperphosphorylated tau which correlated with the appearance of structures that resemble AD characteristic—“straight like filaments” (Hall, et al., Proc. Natl. Acad. Sci. U.S.A. 1997; 94: 4733-4738). In the latter experiments the isoform hyperphosphorylated moiety was, to a larger extent, the long form of h-tau (h-tau₄₂; Hall, et al., Proc. Natl. Acad. Sci. U.S.A. 1997; 94: 4733-4738; Lee, et al., J. Alzheimers Dis. 2009; 16: 99-111). It has been proposed that the physiological function of tau is adversely affected by excess phosphorylation resulting in tau being displaced from microtubules and aggregating, which in turn leads to microtubule disassembly, disruption of axonal transport, and finally synaptic failure (Stamer, et al., J. Cell Biol. 2002; 156: 1051-1063).

Concerning cephalopods, it has been demonstrated that h-tau binds to the squid axonal microtubules, but monomeric h-tau did not affect fast axonal transport (FAT), while filamentous h-tau₄₂ did block anterograde FAT (LaPointe, et al., J. Neurosci. 2009; 87: 440-451). However, other studies have found that flies misexpressing tau show defects in neuronal traffic without evidence of tau aggregation (Jackson, et al., Neuron 2002; 34: 509-519; Mudher, et al., Mol. Psychiatry [In English] 2004; 9:522-530). Finally, extracellular applied h-tau₄₂ to cell cultures produced aberrant signaling through muscarinic receptor activation (Gomez-Ramos, et al., Mol. Cell. Neurosci. 2008; 37: 673-681; Diaz-Hernandez, et al., J. Biol. Chem., 2010; 285: 32539-32548), suggesting that even “normal” tau may be detrimental when its expression becomes elevated or when it accumulates extracellularly. From these observations, it appears that an optimal level of tau phosphorylation is required to achieve the balance in the level of “free” and “microtubule bound” tau that is essential in maintaining microtubule dynamics and subsequent axonal transport.

Kanaan et al., J Neuroscience 2011; 31(27): 9858-9868 report on the role of tau in axonal transport (not synaptic transmission) and shows that the 2-18 N-terminal domain of tau (PAD peptide) is necessary and sufficient for activation of axonal transport in squid axons via the PP1-GSK3 pathway, and that the inhibitor of GSK3 (ING1-35) blocked the inhibition of axonal transport by PAD peptide. Kanaan et al. do not report the effects of phosphorylated tau, PAD or GSK3 in synaptic transmission or vesicle release. Plattner et al., J Biol. Chem. 2006; 281(35): 25457-25465 report that GSK3 is a key mediator of tau hyperphosphorylation and that GSK3 inhibitors would be useful for therapeutic intervention in neurodegenerative taupathies including Alzheimer's Disease (AD). Zhu et al., J Neuroscience 2007; 27(45): 12211-12220 report that activation of GSK3 reduced synaptic transmission, and altered the presynaptic release of neurotransmitter/presynaptic vesicle release and the expression of synapticvesicle associated protein syn1. In addition, Chee et al., Biochemical Society Transactions 2006; 34(1): 88-90 report that disruption of axonal transport and synaptic transmission may be key components of the pathogenic mechanism in tauopathies, and that overexpression of tau disrupts vesicle cycling and synaptic transmission. Moreover, Mandelkow et al., Neurobiology of Aging 2003; 24: 1079-1085 also report that transport of cell organelles and vesicles is inhibited by tau.

### SUMMARY OF THE INVENTION

The present invention is based in part upon the discovery that a tau protein such as h-tau₄₂ is involved in neuronal activity and associated with the pathology of certain neurodegenerative diseases including taupathies. The present invention is further based upon the discovery that inhibiting a tau protein such as h-tau₄₂ may be useful for improving neurotransmission and treating neurodegenerative diseases. Therefore, the present invention provides a novel target for intervention to treat such neurodegenerative diseases including taupathies.

In a first aspect, the present invention provides methods of inhibiting a tau protein or peptide such as h-tau₄₂ or a biologically active fragment, derivative or analog thereof. In some instances the inhibiting is performed decreasing transcription, translation or biological activity of a tau protein or peptide such as h-tau₄₂ or a biologically active fragment, derivative or analog thereof. In some embodiments, the transcription, translation or biological activity of a tau protein such as h-tau₄₂ or a biologically active fragment, derivative or analog thereof may be decreased about 5%, 10%, 20%, 30%, 40%, 50%, 60%, 75%, 80%, 90%, two times, three times, four times, five times, ten times, twenty times, or even fifty or a hundred times less than the transcription, translation or biological activity of a tau protein or peptide such as h-tau₄₂ or a biologically active fragment, derivative or analog thereof in a wild type cell or biological sample. The inhibiting a tau protein or peptide such as h-tau₄₂ or a biologically active fragment, derivative or analog thereof may be performed by administering an effective amount of or a therapeutically effective amount of an agent effective for such inhibiting, including, for instance, one or more of an antibody, a small molecule, a protein, a peptide, or a nucleotide. The administering may be performed in vitro or in vivo, and the administering may be performed by any suitable delivery route such as, for instance, sytemic. The agent may be, for instance, 3-methyladenine, Rapamycin. GSK inhibitor-SB216763, GSK inhibitor-ING-135, LiCl, JNK activator-SB203580, TNT-1 antibody, Xitospongin C, or Dantrolene.

The inhibiting a tau protein or peptide such as h-tau₄₂ or a biologically active fragment, derivative or analog thereof may be effected by reducing phosphorylation or by reducing hyperphosphorylation of the tau protein such as h-tau₄₂ or a biologically active fragment, derivative or analog thereof. Similarly, the inhibiting of a tau protein or peptide such as h-tau₄₂ or a biologically active fragment, derivative or analog thereof may result in and be measured by decreased microtubule disassembly within a neuron, decreased disruption of axonal transport, increased neurotransmitter release, reduced clustering of vesicles, and increased vesicle availability in the active zone of a synapse.

In a second aspect, the present invention provides methods of treating a disease caused all or in part by a tau protein or peptide such as h-tau₄₂ or a biologically active fragment, derivative or analog thereof, by inhibiting a tau protein or peptide such as h-tau₄₂ or a biologically active fragment, derivative or analog thereof or by administering a therapeutically effective amount of an agent effective to inhibit a tau protein or peptide such as h-tau₄₂ or a biologically active fragment, derivative or analog thereof or by decreasing transcription, translation or biological activity of a tau protein or peptide such as h-tau₄₂ or a biologically active fragment, derivative or analog thereof. In some embodiments, the transcription, translation or biological activity of a tau protein or peptide such as h-tau₄₂ or a biologically active fragment, derivative or analog thereof may be decreased 5%, 10%, 20%, 30%, 40%, 50%, 60%, 75%, 80%, 90%, two times, three times, four times, five times, ten times, twenty times, or even fifty or a hundred times less than the transcription, translation or biological activity of a tau protein or peptide such as h-tau₄₂ or a biologically active fragment, derivative or analog thereof in a wild type cell or biological sample. The inhibiting a tau protein or peptide such as h-tau₄₂ or a biologically active fragment, derivative or analog thereof may be performed by administering an effective amount of or a therapeutically effective amount of an agent effective for such inhibiting, including, for instance, one or more of an antibody, a small molecule, a protein, a peptide, or a nucleotide. The administering may be performed in vitro or in vivo, and the administering may be performed by any suitable delivery route such as, for instance, systemic.

Similarly, the agent effective to inhibit a tau protein such as h-tau₄₂ or a biologically active fragment, derivative or analog thereof may be, for instance, an antibody, a small molecule, a protein, a peptide, or a nucleotide. The agent may be, for instance, 3-methyladenine, Rapamycin. GSK inhibitor-SB216763, GSK inhibitor-ING-135, LiCl, INK activator-SB203580, TNT-1 antibody, Xitospongin C, or Dantrolene. The disease caused all or in part by a tau protein or peptide such as h-tau₄₂ or a biologically active fragment, derivative or analog thereof may be, for instance, a neurodegenerative disorder or a neurodegenerative disease. The disorder or disease may result in degeneration of or reduced function of neurons. The disease may be a tauopathy such as, for example, progressive supranuclear palsy, Pick's disease, corticobasal degeneration, frontotemporal dementia with Parkinsonism linked to chromosome 17 (FTDP-17) and Alzheimer's disease (AD). The disease may in some instances be characterized by additional filamentous structures paired helical filaments (PHFs) and straight filaments (SFs) within neurons. The inhibiting a tau protein or peptide such as h-tau₄₂ or a biologically active fragment, derivative or analog thereof may be effected by reducing phosphorylation or by reducing hyperphosphorylation of the tau protein or peptide such as h-tau₄₂ or a biologically active fragment, derivative or analog thereof. Similarly, the inhibiting of a tau protein or peptide such as h-tau₄₂ or a biologically active fragment, derivative or analog thereof may result in and be measured by decreased microtubule disassembly within a neuron, decreased disruption of axonal transport, increased neurotransmitter release, reduced clustering of vesicles, and increased vesicle availability in the active zone of a synapse.

In an third aspect, the present invention provides methods to identify one or more agents such as, for instance, a small molecule, a protein, an antibody, or a nucleotide, that may inhibit a tau protein or peptide such as h-tau₄₂ or a biologically active fragment, derivative or analog thereof. In some embodiments, the transcription, translation or biological activity of a tau protein or peptide such as h-tau₄₂ or a biologically active fragment, derivative or analog thereof may be inhibited or decreased 5%, 10%, 20%, 30%, 40%, 50%, 60%, 75%, 80%, 90%, two times, three times, four times, five times, ten times, twenty times, or even fifty or a hundred times less compared with the transcription, translation or biological activity of tau protein such as h-tau₄₂ or a biologically active fragment, derivative or analog thereof in a wild type cell or biological sample.

This aspect of the invention provides a novel target that can be manipulated to inhibit biological activity of a tau protein such as h-tau₄₂ or a biologically active fragment, derivative or analog thereof or to treat a disease caused all or in part by a tau protein or peptide such as h-tau₄₂ or a biologically active fragment, derivative or analog thereof. Agents such as small molecules, proteins and antibodies may be identified by standard assay techniques known in the art as applied to identify those agents that increase or decrease the biological activity, transcription or expression of a tau protein or peptide such as h-tau₄₂ or a biologically active fragment, derivative or analog thereof. Agents so identified may be useful to treat a disease that may be successfully treated, all or in part, by decreasing biological activity of a tau protein such as h-tau₄₂ or a biologically active fragment, derivative or analog thereof. The disease that may be successfully treated, all or in part, may be, for instance, a neurodegenerative disease or a taupathy. As such, these methods are also methods of screening for therapeutic agents effective to treat a disease caused all or in part by a tau protein or peptide such as h-tau₄₂ or a biologically active fragment, derivative or analog thereof.

The methods for identifying an agent effective to inhibit a tau protein or peptide or a biologically active fragment or derivative or analog thereof may feature administering an agent; and observing either i) a reduction in biological activity of the tau protein or peptide or a biologically active fragment, derivative or analog thereof or ii) a reduction in phosphorylation of the tau protein or peptide or a biologically active fragment, derivative or analog thereof. The inhibiting a tau protein or peptide such as h-tau₄₂ or a biologically active fragment, derivative or analog thereof may be effected by reducing phosphorylation or by reducing hyperphosphorylation of the tau protein or peptide such as h-tau₄₂ or a biologically active fragment, derivative or analog thereof. Similarly, the inhibiting of a tau protein or peptide such as h-tau₄₂ or a biologically active fragment, derivative or analog thereof may result in and be measured by decreased microtubule disassembly within a neuron, decreased disruption of axonal transport, increased neurotransmitter release, reduced clustering of vesicles, and increased vesicle availability in the active zone of a synapse.

In a fourth aspect, the present invention provides pharmaceutical compositions comprising a therapeutically effective amount of an agent effective to inhibit tau protein or peptide such as h-tau₄₂ or a biologically active fragment, derivative or analog thereof in combination with a pharmaceutically acceptable carrier. Such a pharmaceutical composition may be useful for decreasing biological activity of a tau protein or peptide such as h-tau₄₂ or a biologically active fragment, derivative or analog thereof, such as, for instance, by decreasing transcription or translation of a tau protein or peptide such as h-tau₄₂ or a biologically active fragment, derivative or analog thereof or by reducing phosphorylation or hyperphosphorylation of a tau protein or peptide such as h-tau₄₂ or a biologically active fragment, derivative or analog thereof. The agent may be, for instance, 3-methyladenine, Rapamycin. GSK inhibitor-SB216763, GSK inhibitor-ING-135, LiCl, INK activator-SB203580, TNT-1 antibody, Xitospongin C, or Dantrolene.

### BRIEF DESCRIPTION OF THE DRAWINGS

FIG. 1 demonstrates Tau and autophagy and depicts the electrophysiological findings following presynaptic h-tau₄₂ injection. (A) Pre- and post-synaptic potential following a direct electrical stimulation of the presynaptic axon. Synaptic transmission fails following h-tau₄₂ presynaptic injection. (B) Similar results as in (A) following 3-methyladenine injection. (C) Similar results as in (A) following rapamycin injection. (D) Similar results as in (A) following h-tau₄₂ injection in a 3-methyladenine-treated squid. (E) Similar results as in (A) following h-tau₄₂ injection in a rapamycin-treated squid.

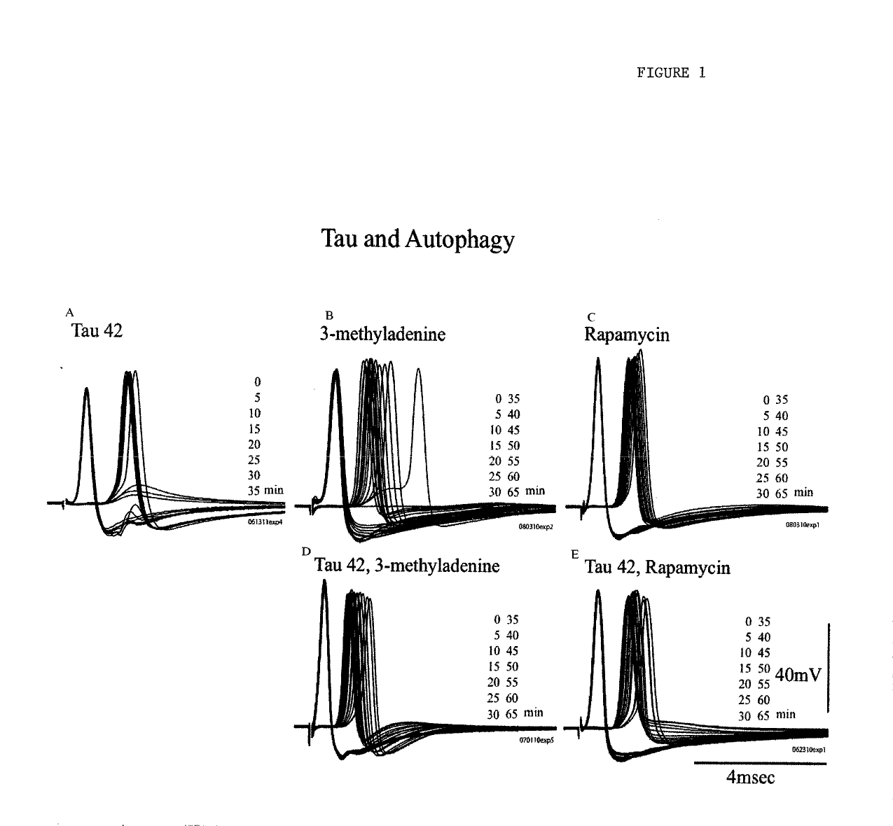

FIG. 2 also depicts the electrophysiological findings following presynaptic h-tau₄₂ injection. (A) demonstrates synaptic transmission following h-tau₄₂ preinjection. (B) Similar results following h-tau₄₂ injection in a SB216763 (GSK3b inhibitor)-treated squid. (C) Similar results as in (A) following h-tau₄₂ injection in a SB203580 (JNK activator)-treated squid. (D) Similar results as in (A) following h-tau₄₂ injection in a ING-135 (GSK3b inhibitor)-treated squid.

FIG. 3 also depicts the electrophysiological findings following presynaptic h-tau₄₂ injection. (A) demonstrates synaptic transmission following h-tau₄₂ preinjection. (B) Similar results following h-tau₄₂ injection in a TN7-1 antibody-treated squid. (C) Similar results as in (A) following h-tau₄₂ injection in a Tau 5 antibody-treated squid.

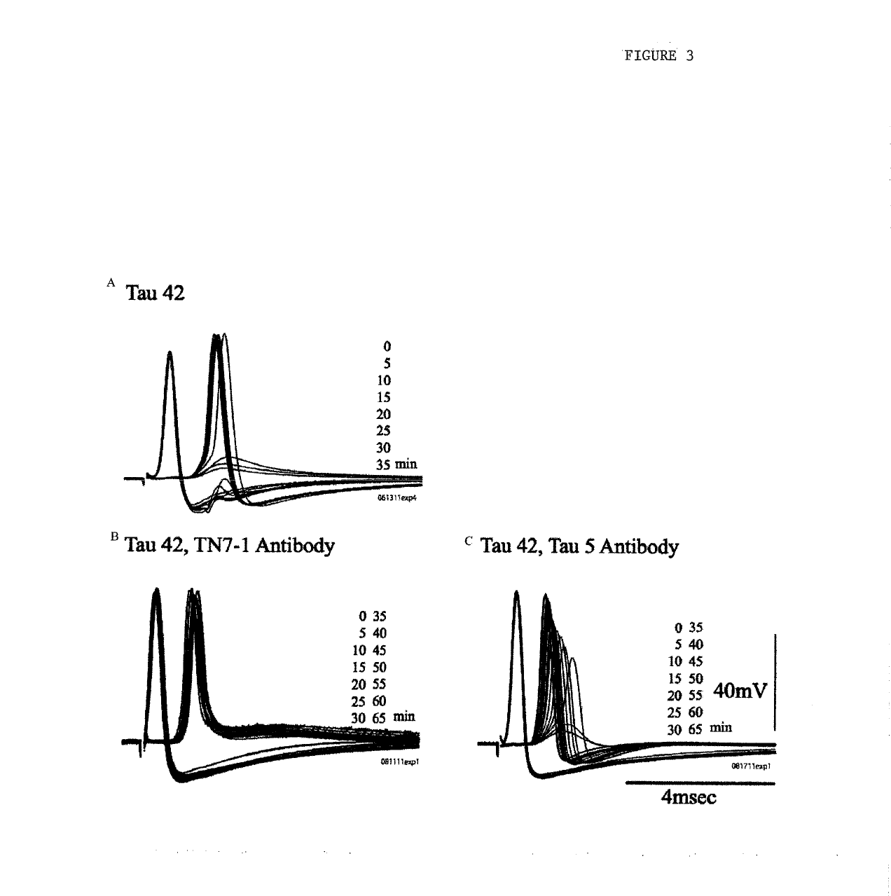

FIG. 4 also depicts the electrophysiological findings following presynaptic h-tau₄₂ injection. (A) Pre- and post-synaptic potential following a direct electrical stimulation of the presynaptic axon. Synaptic transmission fails following h-tau₄₂ preinjection. (B) Similar results as in (A) following 3-methyladenine injection. (C) Similar results as in (A) following rapamycin injection. (D) Similar results as in (A) following h-tau₄₂ injection in a 3-methyladenine-treated squid. (E) Similar results as in (A) following h-tau₄₂ injection in a rapamycin-treated squid.

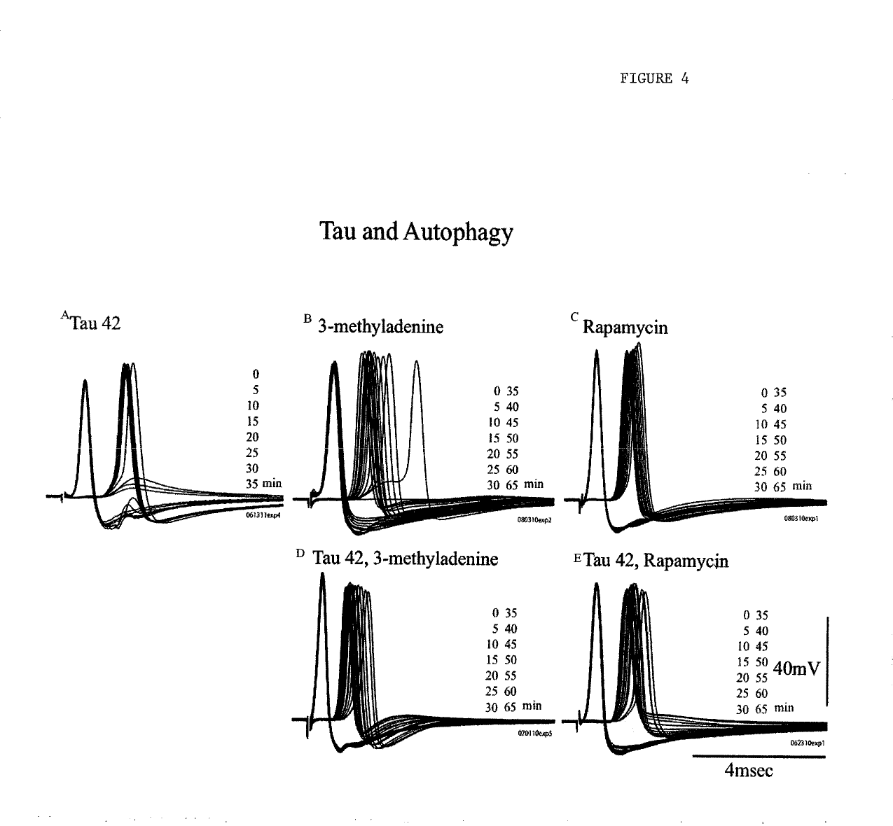

FIG. 5 also depicts the electrophysiological findings following presynaptic h-tau₄₂ injection. (A) Pre- and post-synaptic potential following a direct electrical stimulation of the presynaptic axon. Synaptic transmission fails following h-tau₄₂ preinjection. (B) Similar results as in (A) following h-tau₄₂ injection in a SB216763 (GSK3b inhibitor)-treated squid. (C) Similar results as in (A) following h-tau₄₂ injection in a SB203580 (JNK activator)-treated squid. (D) Similar results as in (A) following h-tau₄₂ injection in a ING-135 (GSK3b inhibitor)-treated squid.

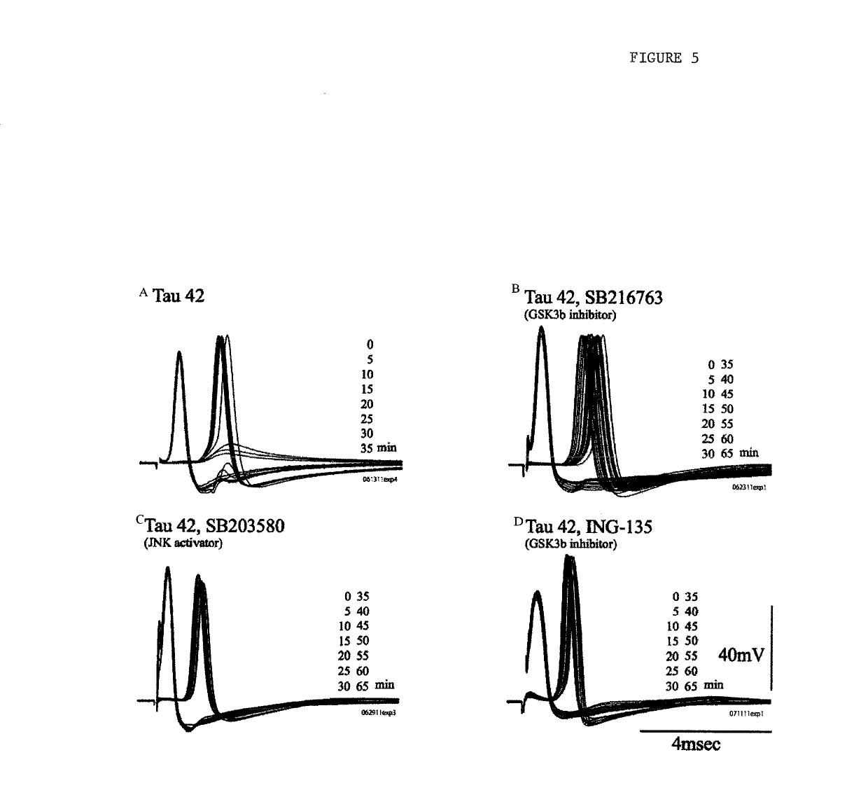

FIG. 6 demonstrates graphically the interaction of a synthetic Tau-PAD peptide to block synaptic transmission by activating GSK3.

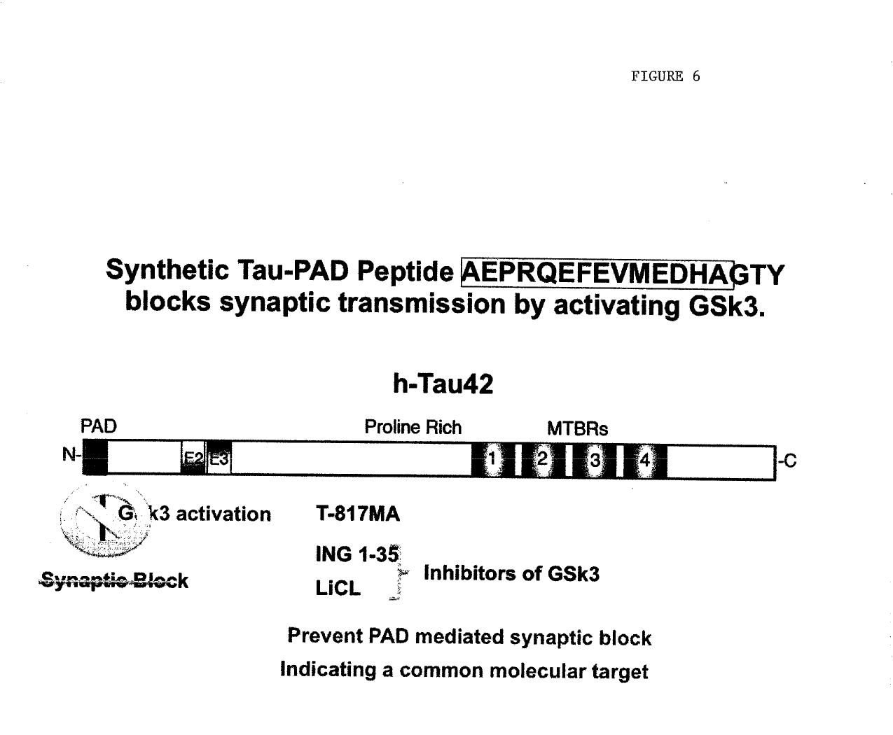

FIG. 7 demonstrates graphically that the PAD domain of h-tau₄₂ is necessary and sufficient to block synaptic transmission.

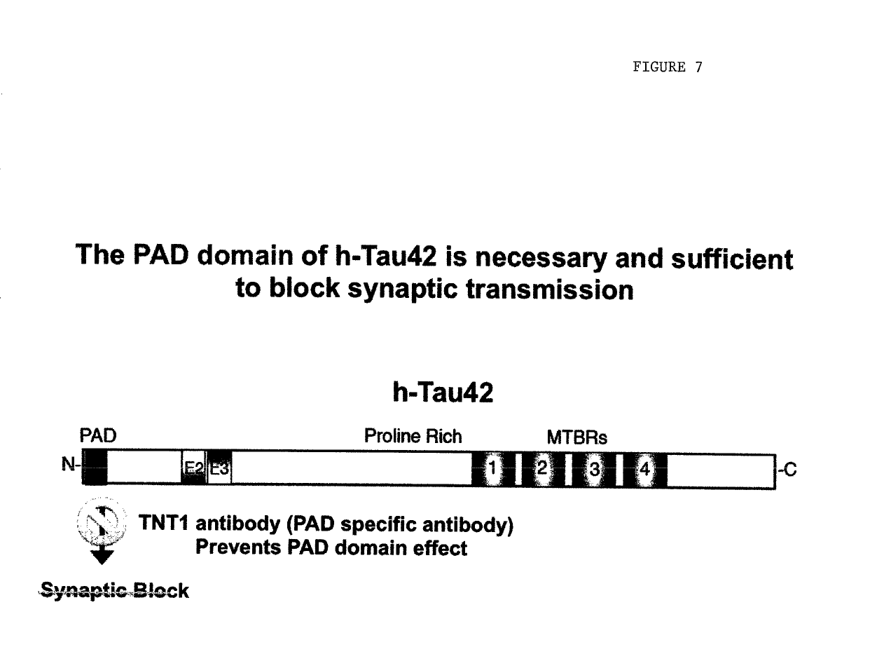

FIG. 8 also depicts the electrophysiological findings following presynaptic h-tau₄₂ injection. (A) Pre- and post-synaptic potential following a direct electrical stimulation of the presynaptic axon. Synaptic transmission fails following h-tau₄₂ preinjection. (B) Similar results as in (A) following h-tau₄₂ injection in a SB216763 (GSK3b inhibitor)-treated squid. (C) Similar results as in (A) following h-tau₄₂ injection in a SB203580 (JNK activator)-treated squid. (D) Similar results as in (A) following h-tau₄₂ injection in a ING-135 (GSK3b inhibitor)-treated squid.

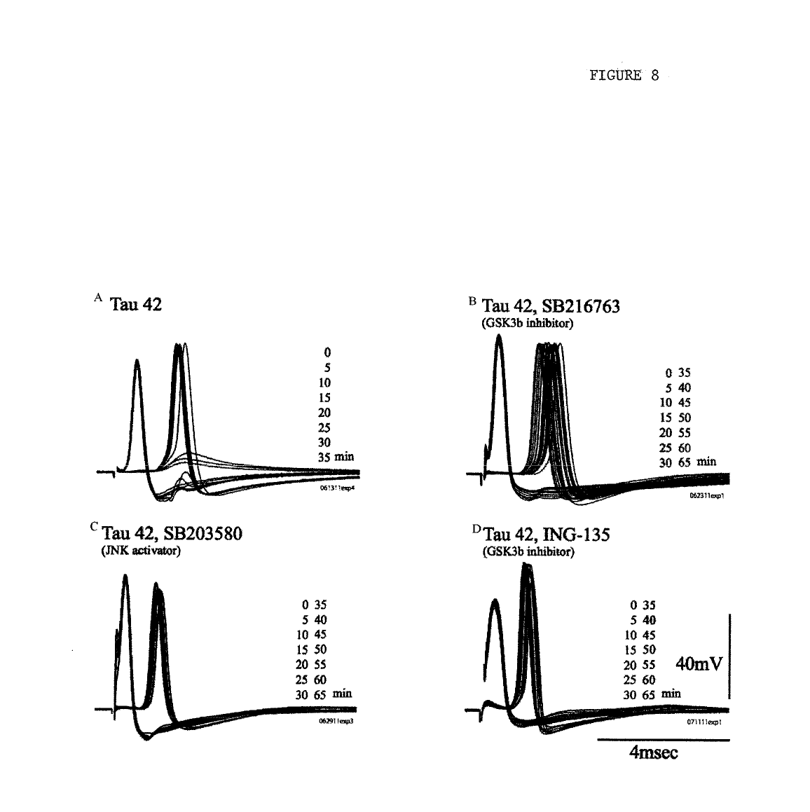

FIG. 9 also depicts the electrophysiological findings following presynaptic h-tau₄₂ injection. (A) Pre- and post-synaptic potential following a direct electrical stimulation of the presynaptic axon. Synaptic transmission fails following h-tau₄₂ preinjection. (B) Similar results as in (A) following h-tau₄₂ injection in a TN7-1 antibody-treated squid. (C) Similar results as in (A) following h-tau₄₂ injection in a Tau 5 antibody-treated squid.

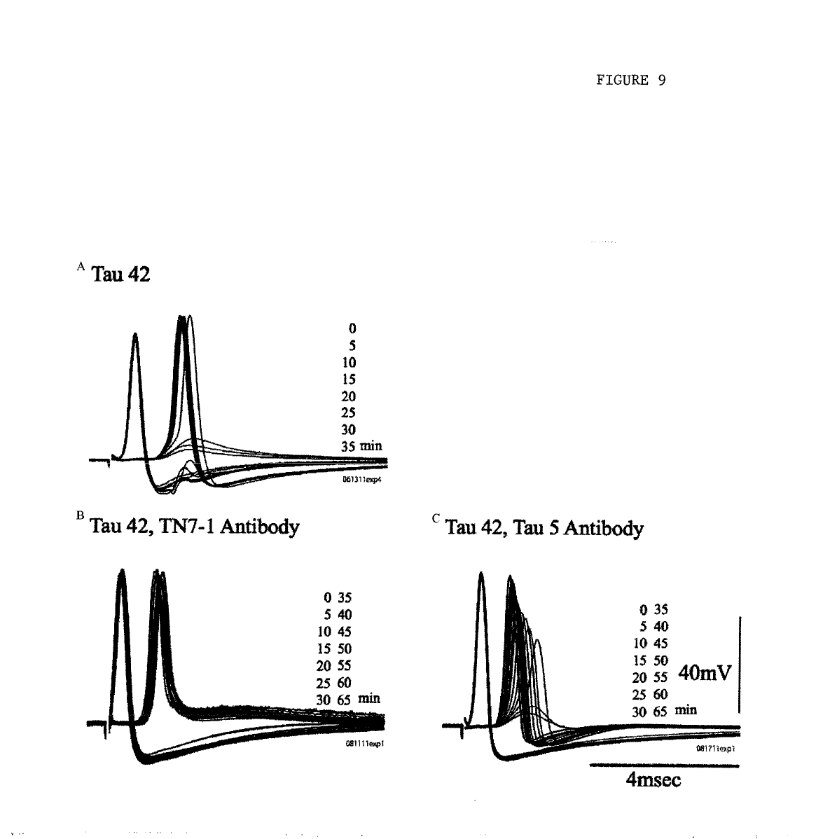

FIG. 10 also depicts the electrophysiological findings following presynaptic h-tau₄₂ injection. (A) Pre- and post-synaptic potential following a direct electrical stimulation of the presynaptic axon. Synaptic transmission fails following h-tau₄₂ preinjection. (B) Similar results as in (A) following h-tau₄₂ injection in a LiCl antibody-treated squid. (C) Similar results as in (A) following h-tau₄₂ injection in a Tau 5 antibody-treated squid.

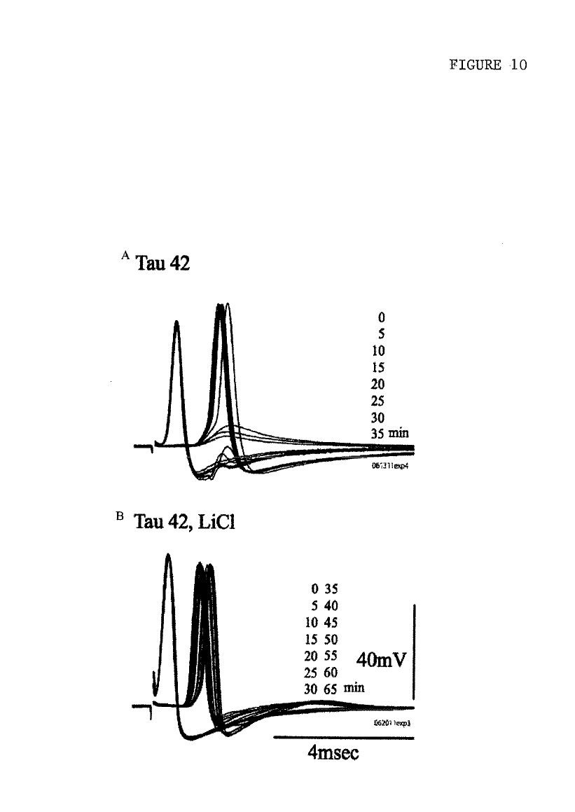

FIG. 11 depicts intracellular Ca2+ storage. (A) Pre- and post-synaptic potential following a direct electrical stimulation of the presynaptic axon. Synaptic transmission fails following h-tau₄₂ preinjection. (B) Similar results as in (A) following h-tau₄₂ injection in a xitospongin C-treated squid. (C) Similar results as in (A) following h-tau₄₂ injection in a dantrolene-treated squid.

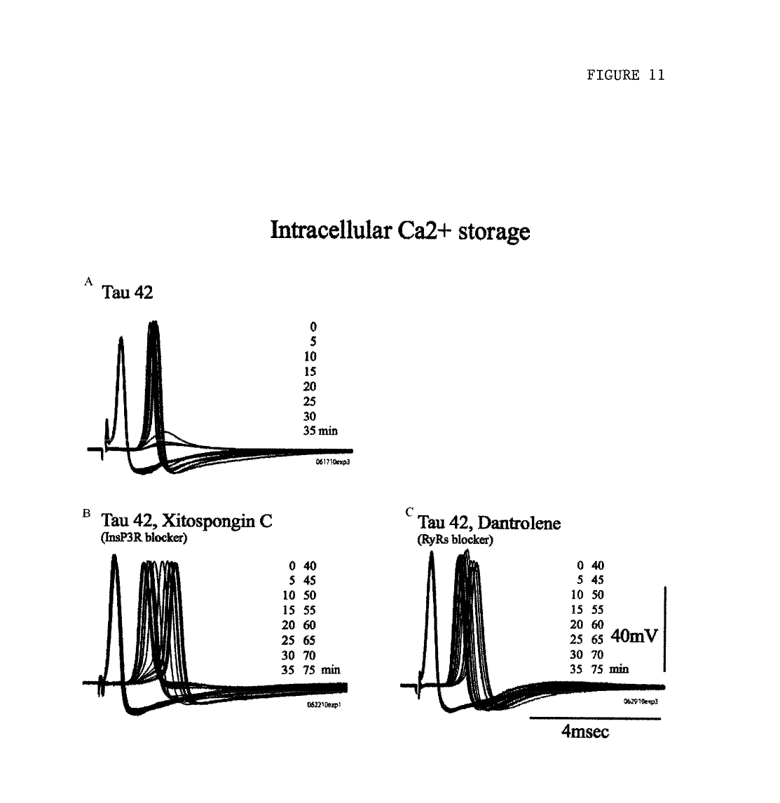

### DETAILED DESCRIPTION OF THE INVENTION

Various terms are used in the specification, which are defined as follows:

By “neurodegenerative disorder or neurodegenerative disease” is meant any disorder or disease resulting in degeneration of or reduced function of neurons. The terms are intended to include tauopathies such as, for example, progressive supranuclear palsy, Pick's disease, corticobasal degeneration, frontotemporal dementia with Parkinsonism linked to chromosome 17 (FTDP-17) and Alzheimer's disease (AD). The terms are intended to include all diseases and disorders that may be characterized by additional filamentous structures paired helical filaments (PHFs) and straight filaments (SFs) within neurons.

A molecule is “antigenic” when it is capable of specifically interacting with an antigen recognition molecule of the immune system, such as an immunoglobulin (antibody) or T cell antigen receptor. An antigenic polypeptide contains at least about 5, and preferably at least about 10, amino acids. An antigenic portion of a molecule can be that portion that is immunodominant for antibody or T cell receptor recognition, or it can be a portion used to generate an antibody to the molecule by conjugating the antigenic portion to a carrier molecule for immunization. A molecule that is antigenic need not be itself immunogenic, i.e., capable of eliciting an immune response without a carrier.

As used herein a “small organic molecule” is an organic compound [or organic compound complexed with an inorganic compound (e.g., metal)] that has a molecular weight of less than 3 kilodaltons, and preferably less than 1.5 kilodaltons. An “agent” of the present invention is preferably a small organic molecule.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition in the host.

In a specific embodiment, the term “about” means within 20%, preferably within 10%, and more preferably within 5%.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Two amino acid sequences are “substantially homologous” when at least about 70% of the amino acid residues, preferably at least about 80%, and most preferably at least about 90, 91, 92, 93, 94, 95, 96, 97, 98, or 99% of the amino acid residues are identical, or represent conservative substitutions. Analogs and derivatives of a protein are normally said to be substantially homologous.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

The terms “a fragment, derivative or analog thereof” refer in some instances to amino acid sequences, peptides and proteins having about 50%, 60%, 70%, 75%, 80%, 85%, 90%, 95%, 96%, 97%, 98%, 99% or about 100% identical sequence to the naturally occurring wild type a tau protein such as h-tau₄₂ protein, such as, for instance, the h-tau₄₂ protein sequence.

The term ‘agent’ means any molecule, including polypeptides, antibodies, polynucleotides, chemical compounds and small molecules. In particular the term agent includes compounds such as test compounds or drug candidate compounds.

The term ‘agonist’ refers to a ligand that stimulates the receptor the ligand binds to in the broadest sense or stimulates a response that would be elicited on binding of a natural ligand to a binding site.

The terms “inhibitor” or “antagonist” refers in some instances to a ligand that stimulates the receptor the ligand binds to in the broadest sense or stimulates a response that would be elicited on binding of a natural ligand to a binding site in instances where the response that is elicited results in reducing or inhibiting the biological activity of its target. The terms “inhibitor” or “antagonist” are intended to encompass agents or molecules that reduce or inhibit the biological activity of another target molecule such as a protein. Such agents or molecules may function by binding to a target molecule such as a protein or may function by reducing the amount of the target molecule such as a protein that is transcribed, translated or expressed. Such agents may be, for instance, small molecules, antibodies or nucleic acids such as, for instance, siRNA, iRNA, etc.

The term ‘assay’ means any process used to measure a specific property of a compound or agent. A ‘screening assay’ means a process used to characterize or select compounds based upon their activity from a collection of compounds.

“Preventing” or “prevention” refers to a reduction in risk of acquiring a disease or disorder.

The term ‘prophylaxis’ is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

The term ‘treating’ or ‘treatment’ of any disease or infection refers, in one embodiment, to ameliorating the disease or infection (i.e., arresting the disease or growth of the infectious agent or bacteria or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease or reducing an infection.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

### H-Tau Affects the Synaptic Release Mechanism

Physiological concentrations of recombinant human tau isoform (full length h-tau₄₂; Perez, et al., Biochemistry 2001; 40: 5983-5991) were directly injected into the presynaptic terminal of a squid giant synapse, to examine possible acute effects of h-tau on the synaptic release mechanism. The results showed that heavy exogenous h-tau₄₂ accumulation induces a rapid and short lasting increase in spontaneous transmitter release followed by a drastic decrease and failure of synaptic transmission. This synaptic block does not affect presynaptic calcium current flow or spike generation at the presynaptic terminal. Immunohistochemistry, performed in h-tau₄₂ injected synapses, demonstrated that h-tau₄₂ becomes phosphorylated rapidly in good temporal agreement with the time course of the transmitter failure. Electron microscopy and electrophysiological experiments unambiguously indicate that h-tau₄₂-mediated synaptic transmission block is due to exocytosis failure. Finally, systemic administration of compound effective to block h-tau₄₂ phosphorylation prevented the structural, biochemical, and functional deleterious effects of h-tau₄₂ microinjection. The data identify several mechanisms of tau-mediated toxicity at the presynaptic terminal, and introduces a potential disease modifier for AD and other tauopathies for which there is no specific treatment presently.

The acute effects of preterminal injection of h-tau₄₂ demonstrate that synaptic dysfunction is an early mechanism in AD and other tauopathies. The squid giant synapse provides unique advantages in addressing the cellular and molecular mechanisms involved in chemical synaptic transmission. The data demonstrate that h-tau₄₂ produces a rapid failure in exocytosis. These results also indicate that h-tau₄₂ has previously unknown physiological properties that are relevant in tau related neurodegenerative process.

Human Tau₄₂ Acutely Blocks Chemical Synaptic Transmission without Affectine Presynaptic Calcium Currents or the Endocytic Pathway.

The data indicate that an excess of h-tau₄₂ protein produces synaptic transmission block by interfering with a mechanism of synaptic vesicle exocytosis. h-tau₄₂ induces a failure in neurotransmitter availability due to reduced synaptic vesicle release, high frequency stimulation, and spontaneous neurotransmitter release data. Moreover, all the h-tau₄₂ injected synapses demonstrate a drastic block of both spontaneous and evoke transmitted release, without affecting presynaptic spike generation or the associated calcium current. These findings reflect the reduced vesicle count at the active zone, the vesicles being instead concentrated in groups away from the active zone. These electron dense vesicular congregations are characterized by profiles resembling vesicular adhesions to microfilaments as would be expected if synapsin 1 were to be dephosphorylated affording a strong adhesion to such microfilaments (Llinás, et al., Proc. Natl. Acad. Sci. U.S.A. 1985; 82: 3035-3039). As a result h-tau₄₂ leads to the failure in exocytosis due to both a defect in the release mechanism and a reduction in vesicular availability (Llinás, et al., J. Physiol. 1991; 436: 257-282).

### Human Tau is Phosphorylated in the Isolated Presynaptic Terminal and Induces Abnormal Vesicular Clustering

Previous experiments in drosophila have shown that misexpression of human-tau (h-tau), the same isoform as the one used in the presently described studies, produced significant neurodegeneration (Jackson, et al., Neuron 2002; 34: 509-519; Avila, et al., Physiol. Rev. 2004; 84: 361-384; Steinhilb, et al., Mol. Biol. Cell 2007; 18: 5060-5068). In the drosophila model tau co-expressed with Shaggy, which generated a single fly homolog of GSK-3β, the phenotype was aggravated (Jackson, et al., Neuron 2002; 34: 509-519). Dysfunctional phenotypes were also found in the central neurons of lamprey, where long-term expression (2-38 days) of several h-tau isoforms produced neurodegenerative changes as a result of accumulation of h-hyperphosphorylated tau which correlated with the appearance of structures that resemble AD characteristic—“straight like filaments” (Hall, et al., Proc. Natl. Acad. Sci. U.S.A. 1997; 94: 4733-4738). In the latter experiments the isoform hyperphosphorylated moiety was, to a larger extent, the long form of h-tau (h-tau₄₂; Hall, et al., Proc. Natl. Acad. Sci. U.S.A. 1997; 94: 4733-4738; Lee, et al., J. Alzheimers Dis. 2009; 16: 99-111). It has been proposed that the physiological function of tau is adversely affected by excess phosphorylation resulting in tau being displaced from microtubules and aggregating, which in turn leads to microtubule disassembly, disruption of axonal transport, and finally synaptic failure (Stamer, et al., J. Cell Biol. 2002; 156: 1051-1063).

Concerning cephalopods, it has been demonstrated that h-tau binds to the squid axonal microtubules, but monomeric h-tau did not affect fast axonal transport (FAT), while filamentous h-tau₄₂ did block anterograde FAT (LaPointe, et al., J. Neurosci. 2009; 87: 440-451). However, other studies have found that flies misexpressing tau show defects in neuronal traffic without evidence of tau aggregation (Jackson, et al., Neuron 2002; 34: 509-519; Mudher, et al., Mol. Psychiatry [In English] 2004; 9:522-530). Finally, extracellular applied h-tau₄₂ to cell cultures produced aberrant signaling through muscarinic receptor activation (Gomez-Ramos, et al., Mol. Cell. Neurosci. 2008; 37: 673-681; Diaz-Hernandez, et al., J. Biol. Chem., 2010; 285: 32539-32548), suggesting that even “normal” tau may be detrimental when its expression becomes elevated or when it accumulates extracellularly. From these observations, it appears that an optimal level of tau phosphorylation is required to achieve the balance in the level of “free” and “microtubule bound” tau that is essential in maintaining microtubule dynamics and subsequent axonal transport.

H-tau₄₂ became phosphorylated in the isolated axon (separated from the cell body) as demonstrated by immunohistochemistry using AT8 antibodies. AT8 recognizes epitopes phosphorylated by GSK3 and cdk5 kinases both of which are found in squid axoplasm (Takahashi, et al., J. Neurosci 1995; 15: 6222-6229; Morfini, et al., EMBO J. 2002; 21: 281-293; Hanger, et al., Expert Rev. Neurother. 2009; 9: 1647-1666), demonstrating that either one or both kinases may be involved in the effects of h-tau₄₂ in the presynaptic terminal. The results demonstrate that isolated axons have the complete machinery to produce local post translational modifications and that these changes may explain, in part, the detrimental effects of excessive “normal tau” on the function of the presynaptic terminal.

Moreover, the vesicle clustering observed in h-tau₄₂ injected synapses, resembled the effect of unphosphorylated synapsin 1 on synaptic vesicle (Jackson, et al., Neuron 2002; 34: 509-519). The fact that h-tau₄₂ is phosphorylated intra-axonally and that unphosphorylated synapsin 1 restrains the vesicle pool to the cytoskeleton—producing a decreased number of vesicles available for exocytosis was actually demonstrated some time ago (Llinás, et al., Proc. Natl. Acad. Sci. U.S.A. 1985; 82: 3035-3039).

H-tau₄₂ apparently induces changes in the balance of kinases and phosphatases, perhaps influenced by the concentration of h-tau aggregates. This may decrease the phosphorylated/dephosphorylated ratio in proteins involved in synaptic vesicle function, such as synapsin 1, which would result in a reduction in the available vesicles and ultimately synaptic transmission failure. Tau is phosphorylated by several protein kinases and this is balanced by protein phosphatases dephosphorylation. The potential kinases and phosphatases involved are so numerous that biochemical experiments dedicated to solve this issue are necessary. This process may involve constitutive vesicular dynamics. A secondary dying-back event (Moreno, et al., Proc. Natl. Acad. Sci. U.S.A. 2009; 106: 5901-5906) may result in the synaptic disconnection encountered in AD pathomorphology.

### Potential Pharmacological Targets of Tau-Mediated Neuropathogenesis

These data demonstrate the protective effect of systemic administration of an agent capable of inhibiting or blocking h-tau₄₂ mediated axonal/synaptic dysfunction. It has been demonstrated that reduction of endogenous tau in an AD mouse model, ameliorates amyloid beta induced neurodegeneration at several levels (Roberson, et al., Science 2007; 316: 750-754). Therefore, an agent capable of inhibiting or blocking tau phosphorylation may treat a tau pathology. Both functional and biochemical h-tau₄₂ induced abnormalities in the presynaptic axon are prevented or ameliorated by these compounds, such as, for instance, 3-methyladenine, Rapamycin. GSK inhibitor-SB216763, GSK inhibitor-ING-135, LiCl, JNK activator-SB203580, TNT-1 antibody, Xitospongin C, or Dantrolene.

These results indicate that h-tau₄₂ affects synaptic release by modifying intracellular phosphorylation homeostasis as a result of h-tau₄₂ hyperphosphorylation. This dynamic change leads to a marked reduction of synaptic vesicle availability, due at least in part to a reduction of synapsin 1 phosphorylation, known to be a powerful modulator of synaptic release (Llinás, et al., Proc. Natl. Acad. Sci. U.S.A. 1985; 82: 3035-3039). Beyond affecting synaptic release the reduction of such vesicular fusion on constitutive vesicular dynamics results in a disconnection event ultimately generating a “dying-back” phenomenon (Stamer, et al., J. Cell Biol. 2002; 156: 1051-1063; Pigino, et al., Proc. Natl. Acad. Sci. U.S.A. 2007; 104: 2442-2447; Serulle, et al., Proc. Natl. acad. Sci. U.S.A., 2007; 104: 2437-2441). Systemic administration of an agent capable of inhibiting or blocking tau phosphorylation, a neuro-protective agent, rescues tau-induced synaptic abnormalities, markedly reduced h-tau₄₂ hyperphosphorylation and prevents synaptic vesicle clustering, as determine by ultrastructural analysis.

In accordance with the present invention conventional molecular biology, microbiology, and recombinant DNA techniques within the skill of the art may be used. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

It should be appreciated that also within the scope of the present invention are DNA sequences encoding a tau protein such as h-tau₄₂ or a biologically active fragment, derivative or analog thereof and comprising or consisting of sequences which are degenerate thereto. DNA sequences having the nucleic acid sequence encoding the peptides of the invention are contemplated, including degenerate sequences thereof encoding the same, or a conserved or substantially similar, amino acid sequence. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC or CUA or CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG or AGU or AGC |
| Proline (Pro or P) | CCU or CCC or CCA or CCG |
| Threonine (Thr or T) | ACU or ACC or ACA or ACG |
| Alanine (Ala or A) | GCU or GCC or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG or AGA or AGG |
| Glycine (Gly or G) | GGU or GGC or GGA or GGG |
| Tryptophan (Trp or W) | UGG |
| Termination codon | UAA (ochre) or UAG (amber) or UGA (opal) |

It should be understood that the codons specified above are for RNA sequences. The corresponding codons for DNA have a T substituted for U.

Mutations can be made in the sequences encoding the protein or peptide sequences of the proteins, peptides or immune activator proteins or peptides of the invention, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

The following is one example of various groupings of amino acids:

Amino Acids with Nonpolar R Groups

### Alanine, Valine, Leucine, Isoleucine, Proline, Phenylalanine, Tryptophan, Methionine

Amino Acids with Uncharged Polar R Groups

### Glycine, Serine, Threonine, Cysteine, Tyrosine, Asparagine, Glutamine

Amino Acids with Charged Polar R Groups (Negatively Charged at pH 6.0)
Aspartic acid, Glutamic acid

### Basic Amino Acids (Positively Charged at pH 6.0)

### Lysine, Arginine, Histidine (at pH 6.0)

Another grouping may be those amino acids with phenyl groups:

### Phenylalanine, Tryptophan, Tyrosine

Another grouping may be according to molecular weight (i.e., size of R groups):

| | | | |
|---|---|---|---|
| Glycine | 75 | Alanine | 89 |
| Serine | 105 | Proline | 115 |
| Valine | 117 | Threonine | 119 |
| Cysteine | 121 | Leucine | 131 |
| Isoleucine | 131 | Asparagine | 132 |
| Aspartic acid | 133 | Glutamine | 146 |
| Lysine | 146 | Glutamic acid | 147 |
| Methionine | 149 | Histidine (at pH 6.0) | 155 |
| Phenylalanine | 165 | Arginine | 174 |
| Tyrosine | 181 | Tryptophan | 204 |

Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

Exemplary and preferred conservative amino acid substitutions include any of: glutamine (Q) for glutamic acid (E) and vice versa; leucine (L) for valine (V) and vice versa; serine (S) for threonine (T) and vice versa; isoleucine (I) for valine (V) and vice versa; lysine (K) for glutamine (Q) and vice versa; isoleucine (I) for methionine (M) and vice versa; serine (S) for asparagine (N) and vice versa; leucine (L) for methionine (M) and vice versa; lysine (L) for glutamic acid (E) and vice versa; alanine (A) for serine (S) and vice versa; tyrosine (Y) for phenylalanine (F) and vice versa; glutamic acid (E) for aspartic acid (D) and vice versa; leucine (L) for isoleucine (I) and vice versa; lysine (K) for arginine (R) and vice versa.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

### Administration of Therapeutic Compositions

According to the present invention, the component or components of a therapeutic composition of the invention may be introduced parenterally, transmucosally, e.g., orally, nasally, or rectally, or transdermally. Preferably, administration is parenteral, e.g., via intravenous injection, and also including, but is not limited to, intra-arteriole, intramuscular, intradermal, subcutaneous, intraperitoneal, intraventricular, and intracranial administration.

In some instances, the components or composition are administered to prevent or treat a neurodegenerative disease and are introduced by injection into the blood. In another embodiment, the therapeutic components or composition can be delivered in a vesicle, in particular a liposome (See, Langer, Science 1990; 249:1527-1533; Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.).

In yet another embodiment, the therapeutic compound can be delivered in a controlled release system. For example, an antibody as described above may be administered using intravenous infusion, an implantable osmotic pump, a transdermal patch, liposomes, or other modes of administration. In one embodiment, a pump may be used (See, Langer, supra; Sefton, CRC Crit. Ref Biomed. Eng. 1987; 14: 201; Buchwald et al., Surgery 1980; 88: 507; Saudek et al., N. Engl. J. Med. 1989; 321: 574). In another embodiment, polymeric materials can be used (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 1983; 23: 61; see also Levy et al., Science 1985; 228: 190; During et al., Ann. Neurol. 1989; 25: 351; Howard et al., J. Neurosurg. 1989; 71:105). In yet another embodiment, a controlled release system can be placed in proximity of a therapeutic target, e.g., the brain, thus requiring only a fraction of the systemic dose (See, e.g., Goodson, in Medical Applications of Controlled Release, supra, vol. 2, pp. 115-138 (1984)). Other controlled release systems are discussed in the review by Langer, Science 1990; 249: 1527-1533.

Thus, a therapeutic composition of the present invention can be delivered by intravenous, intraarterial, intraperitoneal, intramuscular, or subcutaneous routes of administration. Alternatively, the therapeutic composition, properly formulated, can be administered by nasal or oral administration. A constant supply of the therapeutic composition can be ensured by providing a therapeutically effective dose (i.e., a dose effective to induce metabolic changes in a subject) at the necessary intervals, e.g., daily, every 12 hours, etc. These parameters will depend on the severity of the disease or condition being treated, other actions, such as diet modification, that are implemented, the weight, age, and sex of the subject, and other criteria, which can be readily determined according to standard good medical practice by those of skill in the art. A subject in whom administration of the therapeutic composition is an effective therapeutic regiment for a neurodegenerative disease is preferably a human, but can be a primate with a related viral condition. Thus, as can be readily appreciated by one of ordinary skill in the art, the methods and pharmaceutical compositions of the present invention are particularly suited to administration to a number of animal subjects including humans.

Where administration of an antagonist to a tau protein is administered to prevent or treat a neurodegenerative disease, it is preferred for it to be introduced by injection into the blood. The antagonist may be a specific antibody raised against a tau protein such as h-tau₄₂ or a mimic to a tau protein such as h-tau₄₂ that competitively competes with a tau protein such as h-tau₄₂.

In another embodiment, the therapeutic compound can be delivered in a vesicle, in particular a liposome (see Langer, Science 1990; 249: 1527-1533; Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.) To reduce its systemic side effects, this may be a preferred method for introducing an antagonist to a tau protein such as h-tau₄₂.

In yet another embodiment, the therapeutic compound can be delivered in a controlled release system. For example, an antibody as described above may be administered using intravenous infusion, an implantable osmotic pump, a transdermal patch, liposomes, or other modes of administration. In one embodiment, a pump may be used (see Langer, supra; Sefton, CRC Crit. Ref Biomed. Eng. 1987; 14: 201; Buchwald et al., Surgery 1980; 88: 507; Saudek et al., N. Engl. J. Med. 1989; 321: 574). In another embodiment, polymeric materials can be used [see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 1983; 23: 61; see also Levy et al., Science 1985; 228: 190; During et al., Ann. Neurol. 1989; 25: 351; Howard et al., J. Neurosurg. 1989; 71:105). In yet another embodiment, a controlled release system can be placed in proximity of a therapeutic target, e.g., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer [Science 249:1527-1533 (1990)].

Thus, a therapeutic composition of the present invention can be delivered by intravenous, intraarterial, intraperitoneal, intramuscular, or subcutaneous routes of administration. Alternatively, the therapeutic composition, properly formulated, can be administered by nasal or oral administration. A constant supply of the therapeutic composition can be ensured by providing a therapeutically effective dose (i.e., a dose effective to induce metabolic changes in a subject) at the necessary intervals, e.g., daily, every 12 hours, etc. These parameters will depend on the severity of the disease or condition being treated, other actions, such as diet modification, that are implemented, the weight, age, and sex of the subject, and other criteria, which can be readily determined according to standard good medical practice by those of skill in the art.

A subject in whom administration of the therapeutic composition is an effective therapeutic regiment for a neurodegenerative disease is preferably a human, but can be a primate with a related viral condition. Agents that cause an inhibition of a tau protein such as h-tau₄₂ can be used in therapeutic compositions. Thus, as can be readily appreciated by one of ordinary skill in the art, the methods and pharmaceutical compositions of the present invention are particularly suited to administration to a number of animal subjects including humans.

### Transgenic Vectors and Effecting Expression

In one embodiment, a gene encoding a therapeutic compound can be introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus macrophage can be specifically targeted. Examples of particular vectors include, but are not limited to, an attenuated adenovirus vector, such as the vector described by Stratford-Perricaudet et al. J Clin. Invest. 1992; 90:6 26-630), and a defective adeno-associated virus vector (Samulski et al., J. Virol. 1987; 61:3096-3101); Samulski et al., J Virol. 1989; 63: 3822-3828).

In another embodiment the gene or antigene can be introduced in a retroviral vector, e.g., as described in Anderson et al., U.S. Pat. No. 5,399,346; Mann et al., 1983, Cell 33:153; Temin et al., U.S. Pat. No. 4,650,764; Temin et al., U.S. Pat. No. 4,980,289; Markowitz et al., J. Virol. 1988; 62: 1120; Temin et al., U.S. Pat. No. 5,124,263; International Patent Publication No. WO 95/07358, published Mar. 16, 1995, by Dougherty et al.; and Kuo et al., Blood 1993; 82: 845. Targeted gene delivery is described in International Patent Publication WO 95/28494, published October 1995.

Alternatively, the vector can be introduced in vivo by lipofection (Felgner, et. al., Proc. Natl. Acad. Sci. U.S.A. 1987; 84: 7413-7417; see Mackey, et al., Proc. Natl. Acad. Sci. U.S.A. 1988; 85: 8027-8031; Felgner and Ringold, Science 1989; 337: 387-388). Lipids may be chemically coupled to other molecules for the purpose of targeting (See, Mackey, et. al., supra). Targeted peptides, e.g., hormones or neurotransmitters, and proteins such as antibodies, or non-peptide molecules could be coupled to liposomes chemically.

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (See, e.g., Wu et al., J. Biol. Chem. 1992; 267: 963-967; Wu et al., J. Biol. Chem. 1988; 263: 14621-14624; Hartmut et al., Canadian Patent Application No. 2,012,311, filed Mar. 15, 1990).

Antibodies to a Tau Protein Such as h-Tau₄₂

A tau protein such as h-tau₄₂ or a fragment or homolog thereof may be used as an immunogen to generate antibodies that recognize the tau protein such as h-tau₄₂ or a fragment or homolog thereof. Such antibodies include but are not limited to polyclonal, monoclonal, chimeric, single chain, Fab fragments, and a Fab expression library. The tau protein or peptide such as h-tau₄₂ or a fragment or homolog thereof of the invention may be cross reactive, e.g., they may recognize tau protein such as h-tau₄₂ from different species. Polyclonal antibodies may have greater likelihood of cross reactivity. Alternatively, an antibody of the invention may be specific for a single form of tau protein such as h-tau₄₂. Preferably, such an antibody is specific for human tau protein such as h-tau₄₂.

Various procedures known in the art may be used for the production of polyclonal antibodies to tau protein such as h-tau₄₂ or a fragment, derivative or analog thereof. For the production of antibody, various host animals can be immunized by injection with the tau protein such as h-tau₄₂ agent, or a derivative (e.g., fragment or fusion protein) thereof, including but not limited to rabbits, mice, rats, sheep, goats, etc. In one embodiment, the tau protein or peptide agent or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the tau protein such as h-tau₄₂ or a fragment, analog, or derivative thereof, any technique that provides for the production of antibody molecules by continuous cell lines in culture may be used. These include but are not limited to the hybridoma technique originally developed by Kohler and Milstein (Nature 1975; 256: 495-497), as well as the trioma technique, the human B-cell hybridoma technique (Kozbor et al., Immunology Today 1983; 4: 72; Cote et al., Proc. Natl. Acad. Sci. U.S.A. 1983; 80:2026-2030], and the EBV-hybridoma technique to produce human monoclonal antibodies (Cole et al., in Monoclonal Antibodies and Cancer Therapy, Alan R. Liss, Inc., pp. 77-96 (1985)). In an additional embodiment of the invention, monoclonal antibodies can be produced in germ-free animals utilizing recent technology described in PCT/US90/02545. In fact, according to the invention, techniques developed for the production of “chimeric antibodies” (Morrison et al., J. Bacteriol. 1984; 159: 870; Neuberger et al., Nature 1984; 312: 604-608; Takeda et al., Nature 1985; 314: 452-454) by splicing the genes from a mouse antibody molecule specific for a tau protein such as h-tau₄₂ agent together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies (Huston, U.S. Pat. Nos. 5,476,786 and 5,132,405; U.S. Pat. No. 4,946,778) can be adapted to produce tau protein such as h-tau₄₂-specific single chain antibodies. An additional embodiment of the invention utilizes the techniques described for the construction of Fab expression libraries (Huse et al., Science 1989; 246:1275-1281) to allow rapid and easy identification of monoclonal Fab fragments with the desired specificity for a tau protein such as h-tau₄₂ protein, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

### Methods for Screening Drug Libraries

### Identification of Potentially Therapeutic Compounds

Identification and isolation of a gene encoding a tau protein such as h-tau₄₂ of the invention provides for expression of a tau protein such as h-tau₄₂ in quantities greater than can be isolated from natural sources, or in indicator cells that are specially engineered to indicate the activity of a tau protein such as h-tau₄₂ protein expressed after transfection or transformation of the cells. Accordingly, the present invention contemplates a method for identifying agonists and antagonists of a tau protein such as h-tau₄₂ using various screening assays known in the art. In one embodiment, such agonists or antagonists competitively inhibit a tau protein such as h-tau₄₂.

Any screening technique known in the art can be used to screen for antagonists of a tau protein such as h-tau42. The present invention contemplates screens for small molecule ligands or ligand analogs and mimics, as well as screens for natural ligands that bind to and antagonize such activity in vivo. For example, natural products libraries can be screened using assays of the invention for molecules that antagonize a tau protein such as h-tau₄₂. Identification and screening of antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Another approach uses recombinant bacteriophage to produce large libraries. Using the “phage method” (Scott, et al., Science 1990; 249: 386-390; Cwirla, et al., Proc. Natl. Acad. Sci., 1990; 87: 6378-6382; Devlin et al., Science, 1990; 249: 404-406), very large libraries can be constructed. A second approach uses primarily chemical methods, of which the Geysen method (Geysen et al., Molecular Immunology 1986; 23: 709-715; Geysen et al. J. Immunologic Method 1987; 102:259-274) and the method of Fodor et al. Science 1991; 251: 767-773) are examples. Furka et al. 14 th International Congress of Biochemistry, Volume 5, Abstract FR:013 (1988); Furka, Int. J. Peptide Protein Res. 1991; 37:487-493), Houghton (U.S. Pat. No. 4,631,211, issued December 1986) and Rutter et al. U.S. Pat. No. 5,010,175, issued Apr. 23, 1991 describe methods to produce a mixture of peptides that can be tested as agonists or antagonists.

In another aspect, synthetic libraries (Needels et al., Proc. Natl. Acad. Sci. USA 1993; 90: 10700-4; Ohlmeyer et al., Proc. Natl. Acad. Sci. USA 1993; 90:10922-10926; Lam et al., International Patent Publication No. WO 92/00252; Kocis et al., International Patent Publication No. WO 9428028, each of which is incorporated herein by reference in its entirety), and the like can be used to screen for a tau protein such as h-tau₄₂ ligands according to the present invention.

The screening can be performed with recombinant cells that express the tau protein such as h-tau₄₂, or alternatively, using purified protein, e.g., produced recombinantly. For example, the ability of a labeled, soluble or solubilized that includes the ligand-binding portion of the molecule, to bind ligand can be used to screen libraries, as described in the references cited above. In addition, orphan chemokines, potential chemokines, or potential ligands that are obtained from random phage libraries or chemical libraries, as described herein, can be tested by any of the numerous assays well known in the art and exemplified herein. In one particular embodiment of the present invention, an in situ assay is employed in which the detection of the calcium signaling elicited by the binding of a potential chemokine to a chemokine receptor is indicative of the chemokine having specificity for the chemokine receptor, and therefore is a ligand.

### Transgenic Vectors and Inhibition of Expression

In one embodiment, a gene encoding a tau protein such as h-tau₄₂, or antisense or ribozyme specific for a tau protein such as h-tau₄₂ mRNA (termed herein an “antigene”) or a reporter gene can be introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus macrophage can be specifically targeted. Examples of particular vectors include, but are not limited to, a defective herpes virus 1 (HSV1) vector (Kaplitt et al., Molec. Cell. Neurosci. 1991; 2: 320-330), an attenuated adenovirus vector, such as the vector described by Stratford-Perricaudet et al. J Clin. Invest. 1992; 90:626-630, and a defective adeno-associated virus vector (Samulski et al., J. Virol. 1987; 61: 3096-3101; Samulski et al., J. Virol. 1989; 63: 3822-3828).

In another embodiment the gene or antigene can be introduced in a retroviral vector, e.g., as described in Anderson et al., U.S. Pat. No. 5,399,346; Mann et al., 1983, Cell 33:153; Temin et al., U.S. Pat. No. 4,650,764; Temin et al., U.S. Pat. No. 4,980,289; Markowitz et al., J. Virol. 1988; 62: 1120; Temin et al., U.S. Pat. No. 5,124,263; International Patent Publication No. WO 95/07358, published Mar. 16, 1995, by Dougherty et al.; and Kuo et al., Blood 1993; 82: 845. Targeted gene delivery is described in International Patent Publication WO 95/28494, published October 1995.

Alternatively, the vector can be introduced in vivo by lipofection (Felgner, et. al., Proc. Natl. Acad. Sci. U.S.A. 1987; 84: 7413-7417; see Mackey, et al., Proc. Natl. Acad. Sci. U.S.A. 1988; 85: 8027-8031; Felgner, et al., Science 1989; 337: 387-388). Lipids may be chemically coupled to other molecules for the purpose of targeting (see Mackey, et. al., supra). Targeted peptides, e.g., hormones or neurotransmitters, and proteins such as antibodies, or non-peptide molecules could be coupled to liposomes chemically.

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wu et al., J. Biol. Chem. 1992; 267: 963-967; Wu, et al., J. Biol. Chem. 1988; 263: 14621-14624; Hartmut et al., Canadian Patent Application No. 2,012,311, filed Mar. 15, 1990).

As noted above, the present invention extends to the preparation of antisense nucleotides and ribozymes that may be used to interfere with the expression of a tau protein such as h-tau₄₂ at the translational level. This approach utilizes antisense nucleic acid and ribozymes to block translation of a specific mRNA, either by masking that mRNA with an antisense nucleic acid or cleaving it with a ribozyme. Such antisense or ribozyme nucleic acids may be produced chemically, or may be expressed from an “antigene.”

Antisense nucleic acids are DNA or RNA molecules that are complementary to at least a portion of a specific mRNA molecule (see Marcus-Sekura, Anal. Biochem. 1988; 172:298). In the cell, they hybridize to that mRNA, forming a double stranded molecule. The cell does not translate an mRNA in this double-stranded form. Therefore, antisense nucleic acids interfere with the expression of mRNA into protein. Oligomers of about fifteen nucleotides and molecules that hybridize to the AUG initiation codon will be particularly efficient, since they are easy to synthesize and are likely to pose fewer problems than larger molecules when introducing them into organ cells. Antisense methods have been used to inhibit the expression of many genes in vitro (Marcus-Sekura, Anal. Biochem. 1988; 172: 298; Hambor et al., J Exp. Med. 1988; 168: 1237). Preferably synthetic antisense nucleotides contain phosphoester analogs, such as phosphorothiolates, or thioesters, rather than natural phosphoester bonds. Such phosphoester bond analogs are more resistant to degradation, increasing the stability, and therefore the efficacy, of the antisense nucleic acids.

Ribozymes are RNA molecules possessing the ability to specifically cleave other single stranded RNA molecules in a manner somewhat analogous to DNA restriction endonucleases. Ribozymes were discovered from the observation that certain mRNAs have the ability to excise their own introns. By modifying the nucleotide sequence of these RNAs, researchers have been able to engineer molecules that recognize specific nucleotide sequences in an RNA molecule and cleave it (Cech, J. Am. Med. Assoc. 1988; 260: 3030). Because they are sequence-specific, only mRNAs with particular sequences are inactivated.

The DNA sequences encoding the tau protein such as h-tau₄₂ can be used to prepare antisense molecules against and ribozymes that cleave mRNAs for a tau protein such as h-tau₄₂, thus inhibiting expression of the gene encoding the tau protein such as h-tau₄₂, which can reduce the level of HIV translocation in macrophages and T cells.

### DETAILED DESCRIPTION OF THE PREFERRED EMBODIMENTS

### Example 1

### Materials and Methods

### Tau Proteins

Recombinant human tau, h-tau₄₂ (isoform with four tubulin binding motifs and two extra exons in the N-terminal domain) was isolated as previously described (Perez, et al., Biochemistry 2001; 40: 5983-5991).

### Immunohistochemistry

A variation of the array tomography method by Micheva and Smith (2007) was followed. The ganglia were fixed by immersion in 4% paraformaldehyde (EM grade EM Sciences) plus 7.0% sucrose in calcium-free sea water for 3 hours; rinsed with 7% sucrose and 50 mM glycine in 0.1 M PBS; dehydrated with graded ethanol dilutions (50%, 70%, 90%, and 3×100%), embedded in LR White resin (medium grade, SPI), and polymerized in gelatin capsules at 49° C. for 48 hours. Semithin sections (500 nm) were mounted on subbed slides and encircled on the slides with a PAP pen (EM Sciences, USA). The immunocytochemistry was performed as follows: (a) blocked in 50 mM glycine in tris buffer pH 7.6, 5 min; (b) primary antibody incubation, anti-tau PHF (AT8, Thermoscientific, USA) diluted 1:50 in 1% BSA in tris (tris-BSA), 4 h; (c) rinses in tris-BSA 2×5 min: (d) secondary antibody incubation, goat anti-mouse Alexa Fluor 594 diluted 1:150 in tris-BSA, 30 min; (e) tris and distilled water rinses, 4×5 min each; (f) mounting of slides with coverslips and anti-fading mounting media; (g) image under fluorescent microscopy (Zeiss Axioimager, Germany). Controls were performed with the same protocol omitting the primary antibody. Primary antibody: Anti-tau PHF (AT8; Thermoscientific, USA), secondary antibody: Alexa Fluor 594 goat anti-mouse (Invitrogen).

### Administration of Agents Effective for Such Inhibitin Phosphorylation of the Tau Protein

Squid received an oral dose of each agent effective for such inhibiting phosphorylation of the tau protein. At 24 hours after the first dose a second dose was given, and the electrophysiological experiments were performed 1 hour after the second feeding. It was determine that after 24 hours of the double administration, an effective concentration of each agent effective for such inhibiting phosphorylation of the tau protein was measured in the CNS of the gastrically intubated squid (n=10 squid). Synapses from the treated squid were microinjected at the presynaptic axon with h-tau₄₂ (an approximate 80 nM final concentration h-tau₄₂ (n=19), considering a 100× dilution factor. Pre- and post-synaptic potentials were recorded for 90 min.

### Electrophysiology and Microinjections

The squid (Loligo paelli) stellate ganglia isolation from the mantle and the electrophysiological techniques used have been described previously (Llinás, et al., Proc. Natl. Acad. Sci. U.S.A. 1985; 82: 3035-3039). Two glass micropipette electrodes impaled the largest (most distal) presynaptic terminal digit at the synaptic junction site while the post-synaptic axon was impaled by one microelectrode at the junctional site. One of the pre-electrodes was used for pressure microinjection of h-tau₄₂ and also supported voltage clamp current feedback, while the second monitored membrane potential. The total volume injected fluctuated between 0.1 and 1 pl. (Llinás, et al., Proc. Natl. Acad. Sci. U.S.A. 1985; 82: 3035-3039). The exact location of injection and the diffusion and steady-state distribution of the protein/fluorescent dye mix (0.001% dextran fluorescein) were monitored using a fluorescence microscope attached to a Hamamatsu camera system (Middlesex, N.J.). In all experiments a good correlation was observed between the localization of the fluorescence and the electrophysiological findings.

### Electron Microscopy

Immediately following the electrophysiological study the ganglia were removed from the recording chamber, fixed by immersion in glutaraldehyde, post-fixed in osmium tetroxide, stained in block with uranium acetate, dehydrated and embedded in resin (Embed 812, EM Sciences). Ultrathin sections were collected on Pioloform (Ted Pella, Redding, Calif.) and carbon-coated single sloth grids, and contrasted with uranyl acetate and lead citrate. Morphometry and quantitative analysis of the synaptic vesicles were performed with the in house program developed with LabVIEW (National Instruments, Ostin, Tex., USA). Electron micrographs were taken at an initial magnification of ×16,000 and ×31,500 and photographically enlarged to a magnification of ×40,000 and ×79,000 for synaptic vesicles and clathrin-coated vesicle (CCV) counting, respectively. Vesicle density at the synaptic active zones was determined as the number of vesicles per μm², on an average area of 0.8 μm² per active zone. CCV density was determined within the limits of the presynaptic terminal on an average terminal area of 3.3 m2.

### Pharmacological Tools

Each agent effective for such inhibiting phosphorylation of the tau protein was obtained commercially from an appropriate vendor.

### Results

Intra-Axonal h-Tau₄₂ Acutely Blocks Synaptic Transmission

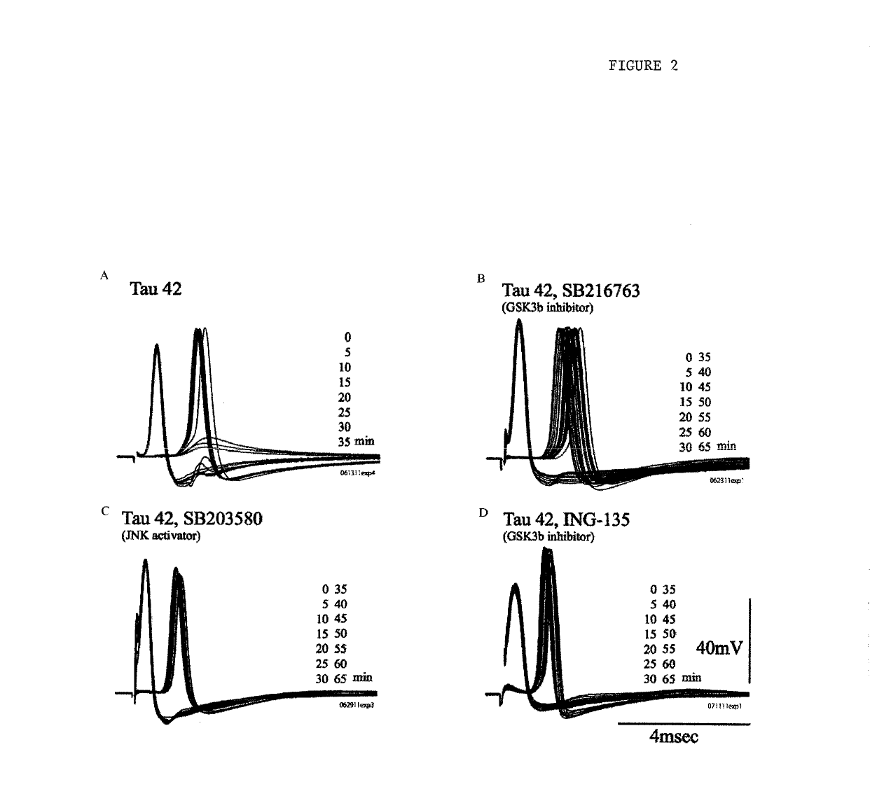

Following presynaptic and post-synaptic axon impalements and the determination of normal synaptic transmission (Llinás, et al., Proc. Natl. Acad. Sci. U.S.A. 1985; 82: 3035-3039; Llinás, et al. Proc. Natl. Acad. Sci. U.S.A. 1994; 91: 12990-12993; Lin, et al., Proc. Natl. Acad. Sci. U.S.A. 1990; 87: 8257-8261), the effect of human tau on synaptic release was evaluated by presynaptic microinjections administered under direct visualization using a fluorescent dye/protein mix, reaching a final concentration after diffusion of approximately 80 nM. (See, Materials and Methods) Presynaptic and post-synaptic potentials were recorded simultaneously under current-clamp configuration. Presynaptic spikes were activated every 5 minutes (low-frequency protocol). With this paradigm, it was determined that 5-10 minutes after an injection of h-tau₄₂, a reduction of transmitter release could be observed. With further time, a total block of transmission resulted, within 30-40 minutes depending on the length of the release zone in the preterminal axon (see, e.g. FIG. 2). No modification of presynaptic spike amplitude or duration ensued. By contrast, following administration of each agent effective for such inhibiting phosphorylation of the tau protein to the squid (see bellow) h-tau₄₂-dependent transmitter block was prevented (see, generally, Figures).

To determine whether the transmission block described above was produced by a reduction of transmitter availability (as would be expected by inhibition of any step in the synaptic vesicle recycling pathway, e.g., endocytosis, refilling of vesicles with transmitter, or docking) or by a defect in synaptic vesicle fusion, the effect of trains of presynaptic high frequency stimuli (100 Hz) was tested. This form of activation rapidly depleted the transmitter, as evidence by the rapid decrease in post-synaptic potential amplitude during a stimulus train. The rapid time course of this decay has been shown to give an estimate of transmitter availability (Llinás, et al. Proc. Natl. Acad. Sci. U.S.A. 1994; 91: 12990-12993), and is a reflection of a decrease in either synaptic vesicle mobilization or docking. On the other hand, synaptic block accompanied by a slow, progressive reduction of post-synaptic amplitude, without amplitude reduction during the tetanic stimulus, is a direct indication that the block is due to a defect in transmitter release (Llinás, et al. Proc. Natl. Acad. Sci. U.S.A. 1994; 91: 12990-12993), i.e., vesicular fusion.

The test paradigm implemented to address this query consisted of synaptic high frequency (100 Hz) spike activation of the presynaptic axon of synapses preinjected with h-tau₄₂ which initially generated post-synaptic repetitive activation. This train stimulation was repeated once a minute until post-synaptic spike failure occurred from the first stimulus in the train. The amplitude of the subthreshold synaptic potentials showed little reduction during the stimulus train itself but steadily reduced in amplitude as h-tau₄₂ mobilizes into the preterminal. The fact that the amplitude of the evoked post-synaptic potentials remained unchanged during the duration of a given repetitive stimulation barrage indicates that the limiting factor was the vesicular release process. This synaptic failure did not recuperate after 15 minutes of rest (as normally occur in this preparation; Llinás, et al., Proc. Natl. Acad. Sci. U.S.A. 1985; 82: 3035-3039; Llinás, et al. Proc. Natl. Acad. Sci. U.S.A. 1994; 91: 12990-12993; Lin, et al., Proc. Natl. Acad. Sci. U.S.A. 1990; 87: 8257-8261), but rather diminished to just noticeable amplitude (Post 3). In synapses from squid pretreated with an agent effective for such inhibiting phosphorylation of the tau protein, microinjection with h-tau₄₂, show the normal reduction of the EPSP amplitude during the repetitive stimulus barrage, however, the treated animals showed normal recovery after a 15-minute rest period. These findings indicate that administering an agent effective for such inhibiting phosphorylation of the tau protein results in the normal recovery of synaptic transmission after high frequency stimulation, indicating that the h-tau₄₂ effects could relate to vesicular availability and not to the actual vesicular release process.

### Tau Modifies Spontaneous Neurotransmitter Release and Produces an Early Transient Increase in Intracellular Calcium

Beyond spike-initiated release, whether h-tau₄₂ may affect spontaneous transmitter release was directly evaluated using post-synaptic noise analysis. A detailed description of the technique has been published by Lin, et al., Proc. Natl. Acad. Sci. U.S.A. 1990; 87: 8257-8261. In all preparations tested (n=6) the membrane noise recorded from the post-synaptic axon increased during the initial 5±1 minute after presynaptic microinjection of h-tau₄₂. This was followed by rapid noise level reduction in parallel with decreased amplitude of the evoked transmitted release. Biphasic changes in noise levels occurred. A frequency spectrum of the membrane noise at different times after the h-tau₄₂ injection occurs. This pattern indicates that block by h-tau₄₂ interferes with both spontaneous and evoked transmitter release.

### Mechanisms Underlying Tau-Induced Synaptic Block

Following the initial finding that transmission is rapidly blocked by h-tau₄₂, whether that this block was associated with changes in presynaptic calcium currents (ICa2+) was tested. The amplitude and time course of ICa2+ were directly determined by presynaptic voltage clamp steps, after blocking voltage dependent K+ and Na+ currents, as previously described (Llinás, et al., Proc. Natl. Acad. Sci. U.S.A. 1985; 82: 3035-3039). ICa²⁺ amplitude and time course were determined at 5-min intervals over a period of 25 minutes following presynaptic injection of h-tau₄₂, 80 nM.

Voltage clamp experiments were implemented with presynaptic voltage steps that generated presynaptic inward calcium current and post-synaptic EPSPs. Following h-tau₄₂ injection presynaptic voltage steps were repeated at 5 minute intervals (low-frequency stimuli), which resulted in a progressive reduction of post-synaptic response amplitude, to total failure, without a change in the amplitude or time course of the presynaptic ICa2+. Under normal conditions this paradigm results in transmitter release that last, unaltered, for up to 2 hours, the maximum period utilized (Llinás, et al., Proc. Natl. Acad. Sci. U.S.A. 1985; 82: 3035-3039). The results show, therefore, that neither the time course nor the amplitudes of the presynaptic calcium currents were altered concomitantly with the transmitter release block induced by h-tau₄₂.

Intra-Axonal h-Tau₄₂ Becomes Phosphorylated and Produces Synaptic Vesicle Aggregation

Since the aggregation of typical tau filaments is accompanied by the development of tau hyperphosphorylation, whether h-tau₄₂ residues serine 202, threonine 205 and/or 231 were phosphorylated in the squid synapse was investigated. AT8 antibodies, as commonly used in neuropathological studies (Goedert, et al., Neurosci. Lett. 1995; 189: 167-169), were used. Immunohistochemistry in a variance of the array tomography technique (Micheva, et al. Neuron 2007; 55: 25-36) was used. Single sections (500 nm) allowed a clear view of the pre- and post-synaptic compartments. Anti-phospho-tau immunohistochemistry was detected as dot-like profiles in the presynaptic compartment in h-tau₄₂ injected synapses but drastically reduced in agent treated squid. These were absent in synapses injected with vehicle. This finding demonstrates that h-tau₄₂ becomes phosphorylated in the squid axon.

Ultrastructural Presynaptic Changes to h-Tau₄₂ Injection

The structural changes that follow h-tau₄₂ injection were addressed by rapidly fixing stellate ganglia (see Materials and Methods) after high or low-frequency stimulation protocols. The material consisted of injected synapses (synaptic active zones from 10 different squid) and vehicle-injected synapses (control, active zones in five synapses). The synapses were fixed ˜72-90 min after h-tau₄₂ injection and processed for ultrastructural microscopy (see Materials and Methods). There was a statistically significant reduction in the number of “docked vesicles” in h-tau₄₂-injected synapses compared to axons injected with vehicle. This reduction was not seen squid treated with an agent effective for such inhibiting phosphorylation of the tau protein following h-tau₄₂ injection.

As shown in representative control synapses, vesicles are normally present at the active zone, some in contact with the presynaptic terminal membrane (docked). By contrast, in h-tau₄₂-injected synapses vesicles were often closely aggregated with electron dense material serving as a bonding matrix (red dot). Similar electron dense material was also observed around vesicles in contact with the active zone (red arrows). At a lower magnification a large number of aggregated vesicular profiles are evident in the vicinity of the active zone (red dots). In squid treated with an agent effective for such inhibiting phosphorylation of the tau protein, the synaptic morphology was quite similar to the vehicle-injected synapses.

Systemic Administration of an Agent Effective for Inhibiting Phosphorylation of the Tau Protein Prevented Tau-Mediated Synaptic Block, Synaptic Vesicle Aggregation, and Decreased h-tau₄₂ Phosphorylation

An agent effective for inhibiting phosphorylation of the tau protein prevents oxidative stress, nitric oxide-induced neurotoxicity, and acts as a neurotrophic factor. Electrophysiologically, no significant changes in the amplitude or time course of the pre- or post-synaptic potentials were observed. Further, ultrastructural studies in synapses used for the electrophysiological experiments demonstrated the number of docked vesicles recovered to the normal range in h-tau₄₂ and an agent effective for inhibiting phosphorylation of the tau protein squid compared to control synapses, with the presence of normal CCV profiles. Also clear was a significant reduction, of electron dense vesicles clusters and electron dense active zones. An agent effective for such inhibiting phosphorylation of the tau protein prevents the h-tau₄₂ dependent synaptic vesicle clustering, indicating a close relation between such morphology and the synaptic transmitter release block observed electrophysiologically. Finally squid pretreated with an agent effective for inhibiting phosphorylation of the tau protein showed a significantly reduced signal of intra-axonal h-tau₄₂ phosphorylation, as detected by AT8 immunohistochemistry.

What is claimed is:

1. A method of inhibiting a tau protein or a biologically active fragment, derivative or analog thereof comprising administering an effective amount of or a therapeutically effective amount of an agent effective for such inhibiting phosphorylation of the tau protein.

2. The method according to claim 1 wherein the tau protein is h-tau₄₂.

3. The method according to claim 1 wherein the agent is selected from the group consisting of 3-methyladenine, rapamycin, GSK inhibitor-SB216763, GSK inhibitor-ING-135, LiCl, INK activator-SB203580, TNT-1 antibody, xitospongin C, and dantrolene.

4. The method according to claim 1 wherein the inhibiting of the tau protein or a biologically active fragment, derivative or analog thereof results in one or more of decreased microtubule disassembly within a neuron, decreased disruption of axonal transport, increased neurotransmitter release, reduced clustering of vesicles, and increased vesicle availability in the active zone of a synapse.

5. A method of treating a disease caused all or in part by a tau protein or peptide or a biologically active fragment, derivative or analog thereof comprising administering a therapeutically effective amount of an agent effective to inhibit a tau protein or peptide or a biologically active fragment, derivative or analog thereof.

6. The method according to claim 5 wherein the tau protein is h-tau₄₂.

7. The method according to claim 5 wherein the agent is selected from the group consisting of 3-methyladenine, rapamycin, GSK inhibitor-SB216763, GSK inhibitor-ING-135, LiCl, INK activator-SB203580, TNT-1 antibody, xitospongin C, and dantrolene.

8. The method according to claim 5 wherein the inhibiting of the tau protein or a biologically active fragment, derivative or analog thereof results in one or more of decreased microtubule disassembly within a neuron, decreased disruption of axonal transport, increased neurotransmitter release, reduced clustering of vesicles, and increased vesicle availability in the active zone of a synapse.

9. The method according to claim 5 wherein the disease caused all or in part by a tau protein or a biologically active fragment, derivative or analog thereof is a neurodegenerative disease.

10. The method according to claim 5 wherein the disease caused all or in part by a tau protein is a tauopathy.

11. The method according to claim 10 wherein the taupathy is selected from the group consisting of a progressive supranuclear palsy, Pick's disease, corticobasal degeneration, frontotemporal dementia with Parkinsonism linked to chromosome 17 (FTDP-17) and Alzheimer's disease (AD).

12. The method according to claim 5 wherein inhibiting a tau protein such as h-tau₄₂ or a biologically active fragment, derivative or analog thereof results in reducing phosphorylation or of the tau protein or a biologically active fragment, derivative or analog thereof.

13. A method for identifying an agent effective to inhibit a tau protein or a biologically active fragment, derivative or analog thereof comprising:
a) administering an agent; and
b) observing either i) a reduction in biological activity of the tau protein or a biologically active fragment, derivative or analog thereof or ii) a reduction in phosphorylation of the tau protein or a biologically active fragment, derivative or analog thereof.

14. The method of claim 13 wherein the agent is selected from the group consisting of a small molecule, a protein, an antibody and a nucleotide.

15. The method of claim 13 wherein the tau protein is h-tau₄₂.

16. The method of claim 13 wherein the agent reduces phosphorylation of the tau protein or a biologically active fragment, derivative or analog thereof.

17. The method of claim 13 wherein the observing the reduction in biological activity of the tau protein or a biologically active fragment, derivative or analog thereof is performed by observing one or more of decreased microtubule disassembly within a neuron, decreased disruption of axonal transport, increased neurotransmitter release, reduced clustering of vesicles, and increased vesicle availability in the active zone of a synapse.

18. A pharmaceutical composition comprising a therapeutically effective amount of an agent effective to inhibit a tau protein or a biologically active fragment, derivative or analog thereof in combination with a pharmaceutically acceptable carrier.

19. The pharmaceutical composition according to claim 18 wherein the agent is selected from the group consisting of 3-methyladenine, rapamycin, GSK inhibitor-SB216763, GSK inhibitor-ING-135, LiCl, INK activator-SB203580, TNT-1 antibody, xitospongin C, and dantrolene.


SEQUENCE LISTING (1 sequence)

SEQ ID NO: 1 - protein, 17 aa, Artificial Sequence
  synthetic
AEPRQEFEVMEDHAGTY